(19) United States
(12) Patent Application Publication
(10) Pub. No.: US 2026/0141981 A1
(43) Pub. Date: May 21, 2026

(54) METHODS AND SYSTEMS FOR PERFORMING GENOMIC VARIANT CALLS BASED ON IDENTIFIED OFF-TARGET SEQUENCE READS

(71) Applicant: Foundation Medicine, Inc., Boston, MA (US)

(72) Inventor: Dean PAVLICK, Cambridge, MA (US)

(73) Assignee: Foundation Medicine, Inc., Boston, MA (US)

(21) Appl. No.: 19/120,957

(22) PCT Filed: Oct. 13, 2023
(86) PCT No.: PCT/US2023/076805

§ 371 Date: Apr. 14, 2025

Related U.S. Application Data
(60) Provisional application No. 63/416,392, filed on Oct. 14, 2022.

(51) Int. Cl. G16B 30/10; C12Q 1/6855; C12Q 1/6874; G16B 20/20; G16H 15/00; G16H 50/20

(52) U.S. Cl. CPC G16B 30/10; C12Q 1/6855; C12Q 1/6874; G16B 20/20; G16H 15/00; G16H 50/20

(57) ABSTRACT

Methods for identifying off-target sequence reads and performing genomic variant calls based on the identified off-target sequence reads are described. The method includes receiving sequence read data for a sample derived from a subject, determining one or more on-target sequence reads and one or more off-target sequence reads based on the sequence read data, and identifying one or more intervals of off-target sequence read data based on the one or more off-target sequence reads. The method further includes determining one or more off-target sequence reads to be included in the one or more on-target sequence reads, modifying the one or more on-target sequence reads to include the one or more intervals of sequence read data associated with the off-target sequence reads determined to be included in the on-target sequence reads, and performing a genomic variant call utilizing the modified one or more on-target sequence reads.

### CROSS-REFERENCE TO RELATED APPLICATIONS

This application claims the priority benefit of U.S. Provisional Patent Application Ser. No. 63/416,392, filed Oct. 14, 2022, the contents of which are incorporated herein by reference in their entirety.

### FIELD OF THE INVENTION

The present disclosure relates generally to genomic variant calls, and, more particularly, to methods and systems for identifying off-target sequence reads and performing genomic variant calls based on identified off-target sequence reads.

### BACKGROUND

Genomic variant calling may generally include identifying single nucleotide polymorphisms (SNPs) and small insertions or deletions from next generation sequencing (NGS) genomic data. Somatic mutations, such as point mutations, insertions, and deletions accumulate in the genome and have the potential use for stratification of disease (e.g. cancer), and as biomarkers for disease diagnosis, prognosis, and prediction of treatment outcomes. However, full genome analysis for individual patient's diseased tissue is expensive, time consuming, and inefficient. Thus, groups have developed protocols to increase efficiency in testing for mutations in genes that have previously been associated with disease. Even with more advanced baiting and sequencing protocols, these methods produce “off-target sequence reads” or reads corresponding to a genomic locus that is not targeted by a bait molecule or other locus-specific enrichment technique used in a targeted sequencing assay. Off-target sequencing reads are often discarded in analysis pipelines, but may still be important for disease stratification.

### BRIEF SUMMARY OF THE INVENTION

Disclosed herein are methods and systems for identifying off-target sequence reads of interest and performing genomic variant calls based on the identified off-target sequence reads. In certain embodiments, one or more processors of a genomic variant calling system may receive sequence read data for a sample derived from a subject. In certain embodiments, the one or more processors may then determine one or more on-target sequence reads and one or more off-target sequence reads based on the sequence read data. In certain embodiments, the one or more processors may identify one or more intervals of off-target sequence read data based on the one or more off-target sequence reads. In certain embodiments, the one or more processors may then determine one or more off-target sequence reads to be included in the one or more on-target sequence reads. In certain embodiments, the one or more processors may modify the one or more on-target sequence reads to include the one or more intervals of sequence read data associated with the off-target sequence reads determined to be included in the on-target sequence reads. In certain embodiments, the one or more processors may then perform a genomic variant call utilizing the modified one or more on-target sequence reads.

In certain embodiments, one or more processors of a genomic variant calling system may receive sequence read data for a sample derived from a subject. In certain embodiments, the one or more processors may then determine one or more on-target sequence reads and one or more off-target sequence reads based on the sequence read data. In certain embodiments, the one or more processors may then identify, based on the one or more off-target sequence reads, one or more intervals of off-target sequence read data corresponding to an enhancer of zest homologs inhibitory protein (EZHIP). In certain embodiments, the one or more processors may then modify the one or more on-target sequence reads to include the one or more intervals of off-target sequence read data corresponding to the EZHIP. In certain embodiments, the one or more processors may then perform a genomic variant call utilizing the modified one or more on-target sequence reads.

In certain embodiments, a genomic variant calling system may obtain at least one sample from an individual. In certain embodiments, the genomic variant calling system may isolate nucleic acids from the at least one sample. In certain embodiments, the genomic variant calling system sequence the isolated nucleic acids to produce a plurality of sequence reads. In certain embodiments, one or more processors of the genomic variant calling system may receive sequence read data based on the plurality of sequence reads. In certain embodiments, the one or more processors may then determine one or more on-target sequence reads and one or more off-target sequence reads based on the sequence read data. In certain embodiments, the one or more processors may identify one or more intervals of off-target sequence read data based on the one or more off-target sequence reads. In certain embodiments, the one or more processors may then determine one or more off-target sequence reads to be included in the one or more on-target sequence reads. In certain embodiments, the one or more processors may modify the one or more on-target sequence reads to include the one or more intervals of sequence read data associated with the off-target sequence reads determined to be included in the on-target sequence reads. In certain embodiments, the one or more processors may then perform a genomic variant call utilizing the modified one or more on-target sequence reads.

In certain embodiments, a genomic variant calling system may provide a plurality of nucleic acid molecules obtained from a sample. In certain embodiments, the genomic variant calling system may then ligate one or more adapters onto one or more nucleic acid molecules from the plurality of nucleic acid molecules. In certain embodiments, the genomic variant calling system may amplify the one or more ligated nucleic acid molecules from the plurality of nucleic acid molecules. In certain embodiments, the genomic variant calling system may capture amplified nucleic acid molecules from the amplified nucleic acid molecules. In certain embodiments, the genomic variant calling system may then sequence, by a sequencer, the captured nucleic acid molecules to obtain a plurality of sequence reads that represent a set of subgenomic intervals in the nucleic acid molecules. In certain embodiments, one or more processors of the genomic variant calling system may receive sequence read data based on the plurality of sequence reads. In certain embodiments, the one or more processors may determine one or more on-target sequence reads and one or more off-target sequence reads based on the sequence read data. In certain embodiments, the one or more processors may then identify one or more intervals of off-target sequence read data based on the one or more off-target sequence reads. In certain embodiments, the one or more processors may determine one or more off-target sequence reads to be included in the one or more on-target sequence reads. In certain embodiments, the one or more processors may modify the one or more on-target sequence reads to include the one or more intervals of sequence read data associated with the off-target sequence reads determined to be included in the on-target sequence reads. In certain embodiments, the one or more processors may perform a genomic variant call utilizing the modified one or more on-target sequence reads.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates a genomic variant calling system for identifying one or more off-target sequence reads of interest and performing genomic variant calls based on the identified off-target sequence reads.

FIG. 2 illustrates a genomic variant calling system for identifying one or more off-target sequence reads of interest and performing genomic variant calls based on the identified off-target sequence reads.

FIG. 3 illustrates an exemplary diagram of one or more example on-target sequence reads and off-target sequence reads.

FIG. 4A illustrates a flow diagram of an exemplary method for identifying one or more off-target sequence reads of interest and performing genomic variant calls based on the identified off-target sequence reads.

FIG. 4B illustrates a flow diagram of an exemplary method for identifying one or more off-target sequence reads corresponding to EZHIP and performing genomic variant calls based on the identified off-target sequence reads.

FIG. 5 illustrates another flow diagram of an exemplary method for identifying one or more off-target sequence reads of interest and performing genomic variant calls based on the identified off-target sequence reads.

FIG. 6 illustrates an example computing system, according to some embodiments.

### DETAILED DESCRIPTION

Methods and systems for identifying off-target sequence reads of interest and performing genomic variant calls based on identified off-target sequence reads. In certain embodiments, one or more processors of a genomic variant calling system may receive sequence read data for a sample derived from a subject. In certain embodiments, the one or more processors may then determine one or more on-target sequence reads and one or more off-target sequence reads based on the sequence read data. In certain embodiments, the one or more processors may identify one or more intervals of off-target sequence read data based on the one or more off-target sequence reads. In certain embodiments, the one or more processors may then determine one or more off-target sequence reads to be included in the one or more on-target sequence reads. In certain embodiments, the one or more processors may modify the one or more on-target sequence reads to include the one or more intervals of sequence read data associated with the off-target sequence reads determined to be included in the on-target sequence reads. In certain embodiments, the one or more processors may then perform a genomic variant call utilizing the modified one or more on-target sequence reads.

In certain embodiments, the one or more processors may perform a quality control process based on the genomic variant call. In certain embodiments, the one or more processors may generate a report based on the genomic variant call. In certain embodiments, prior to receiving the sequence read data, the one or more processors may perform a hybridization-capture process to extract genomic reads for next-generation sequencing (NGS), and to perform the NGS to generate a plurality of base-call sequencing data based on the extracted genomic reads. In certain embodiments, the one or more processors may further convert the plurality of base-call sequencing data into a sorted binary alignment map (BAM) file. In certain embodiments, receiving sequence read data may include receive sequence reads corresponding to a targeted 324 genes. In certain embodiments, the one or more processors may generate a reference file of intervals of sequence read data based on the targeted 324 genes and the one or more intervals of off-target sequence read data. In certain embodiments, the one or more processors may perform a genomic variant call utilizing the reference file.

In certain embodiments, prior to modifying the one or more on-target sequence reads to include the one or more intervals of off-target sequence read data, the one or more processors may perform a genomic variant call utilizing the one or more on-target sequence reads. In certain embodiments, prior to modifying the one or more on-target sequence reads to include the one or more intervals of off-target sequence read data, the one or more processors may perform a genomic variant call utilizing the one or more off-target sequence reads, the genomic variant call corresponding to the one or more intervals of off-target sequence read data. In certain embodiments, identifying the one or more intervals of off-target sequence read data may include identifying one or more base-pair (bp) intervals outside of a predetermined bp interval. In certain embodiments, performing the genomic variant call utilizing the modified one or more on-target sequence reads may include identifying an indication of a genetic biomarker of the sample.

In certain embodiments, the genetic biomarker of the sample may include an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3(NTRK 3 ) gene alteration, a fibroblast growth factor receptor 2(FGFR2 ) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3. In certain embodiments, the sample may include a tumor. In certain embodiments, the tumor may include at least one of a diffuse midline glioma (DMG), H3K27-mutant tumor, a posterior fossa ependymoma group PFA (PFA-EFN) tumor, a DMG, H3K27-WT tumor with enhancer of zest homologs inhibitory protein (EZHIP) overexpression, a wingless-activated (WNT) medulloblastoma tumor, an atypical teratoid/rhabdoid tumor (AT/RT), or a germinoma tumor.

In certain embodiments, the one or more processors may further comprising identify one or more intervals of off-target sequence read data corresponding to one or more of an enhancer of zest homologs inhibitory protein (EZHIP), H3 lysine 27 hypo-trimethylation (H3K27me3), or HIST1H3B K27M. In certain embodiments, performing the genomic variant call may include performing a genotypic or phenotypic call utilizing the modified one or more on-target sequence reads. In some embodiments, the off-target sequence reads may include less than 50%, less than 40%, less than 30%, less than 20%, or less than 10% of the sequence reads in the sequence read data. In certain embodiments, the one or more processors may cause one or more electronic devices to display a report generated based on the genomic variant call. In certain embodiments, causing the one or more electronic devices to display the report may include causing a human machine interface (HMI) associated with a pathologist or a clinician to display the report.

The disclosed methods and systems thus identify off-target sequence reads of interest and perform genomic variant calls based on the identified off-target sequence reads.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The terms “clinically relevant” and “clinically significant” are used interchangeably and refer to any information relevant to diagnosis and/or treatment of a disease (e.g., cancer) of a patient, including but not limited to information that may be of a highest priority or relevance to the diagnosis and/or treatment of the disease based on a disease state of the patient.

The term “medical information” refers to one or more therapeutic, diagnostic, prognostic, potential germline, potential clonal hematopoiesis, or other related information based, at least in part, on a patient's medical information.

The section headings used herein are for organizational purposes only and are not to be construed as limiting the subject matter described.

### Methods for Identifying Off-Target Sequence Reads and Performing Genomic Variant Calls Utilizing the Identified Off-Target Sequence Reads

The disclosed methods are directed toward identifying off-target sequence reads of interest and performing genomic variant calls based on identified off-target sequence reads. In certain embodiments, one or more processors of a genomic variant calling system may receive sequence read data for a sample derived from a subject. In certain embodiments, the one or more processors may then determine one or more on-target sequence reads and one or more off-target sequence reads based on the sequence read data. In certain embodiments, the one or more processors may identify one or more intervals of off-target sequence read data based on the one or more off-target sequence reads. In certain embodiments, the one or more processors may then determine one or more off-target sequence reads to be included in the one or more on-target sequence reads. In certain embodiments, the one or more processors may modify the one or more on-target sequence reads to include the one or more intervals of sequence read data associated with the off-target sequence reads determined to be included in the on-target sequence reads. In certain embodiments, the one or more processors may then perform a genomic variant call utilizing the modified one or more on-target sequence reads.

In certain embodiments, the one or more processors may perform a quality control process based on the genomic variant call. In certain embodiments, the one or more processors may generate a report based on the genomic variant call. In certain embodiments, prior to receiving the sequence read data, the one or more processors may perform a hybridization-capture process to extract genomic reads for next-generation sequencing (NGS), and to perform the NGS to generate a plurality of base-call sequencing data based on the extracted genomic reads. In certain embodiments, the one or more processors may further convert the plurality of base-call sequencing data into a sorted binary alignment map (BAM) file. In certain embodiments, receiving sequence read data may include receive sequence reads corresponding to a targeted 324 genes. In certain embodiments, the one or more processors may generate a reference file of intervals of sequence read data based on the targeted 324 genes and the one or more intervals of off-target sequence read data. In certain embodiments, the one or more processors may perform a genomic variant call utilizing the reference file.

In certain embodiments, prior to modifying the one or more on-target sequence reads to include the one or more intervals of off-target sequence read data, the one or more processors may perform a genomic variant call utilizing the one or more on-target sequence reads. In certain embodiments, prior to modifying the one or more on-target sequence reads to include the one or more intervals of off-target sequence read data, the one or more processors may perform a genomic variant call utilizing the one or more off-target sequence reads, the genomic variant call corresponding to the one or more intervals of off-target sequence read data. In certain embodiments, identifying the one or more intervals of off-target sequence read data may include identifying one or more base-pair (bp) intervals outside of a predetermined bp interval. In certain embodiments, performing the genomic variant call utilizing the modified one or more on-target sequence reads may include identifying an indication of a genetic biomarker of the sample.

In certain embodiments, the genetic biomarker of the sample may include an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3(NTRK 3 ) gene alteration, a fibroblast growth factor receptor 2(FGFR2 ) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3. In certain embodiments, the sample may include a tumor. In certain embodiments, the tumor may include at least one of a diffuse midline glioma (DMG), H3K27-mutant tumor, a posterior fossa ependymoma group PFA (PFA-EFN) tumor, a DMG, H3K27-WT tumor with enhancer of zest homologs inhibitory protein (EZHIP) overexpression, a wingless-activated (WNT) medulloblastoma tumor, an atypical teratoid/rhabdoid tumor (AT/RT), or a germinoma tumor.

In certain embodiments, the one or more processors may further comprising identify one or more intervals of off-target sequence read data corresponding to one or more of an enhancer of zest homologs inhibitory protein (EZHIP), H3 lysine 27 hypo-trimethylation (H3K27me3), or HISTIH3B K27M. In certain embodiments, performing the genomic variant call may include performing a genotypic or phenotypic call utilizing the modified one or more on-target sequence reads. In some embodiments, the off-target sequence reads may include less than 50%, less than 40%, less than 30%, less than 20%, or less than 10% of the sequence reads in the sequence read data. In certain embodiments, the one or more processors may cause one or more electronic devices to display a report generated based on the genomic variant call. In certain embodiments, causing the one or more electronic devices to display the report may include causing a human machine interface (HMI) associated with a pathologist or a clinician to display the report.

In certain embodiments, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In certain embodiments, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof. In other embodiments, the disclosed methods may be used to identify variants other than ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof. For example, these variants may be based on targeted genes, whereas variants on non-targeted genes may also be identified using the disclosed methods.

In certain embodiments, one or more processors of a genomic variant calling system may receive sequence read data for a sample derived from a subject. In certain embodiments, the one or more processors may then determine one or more on-target sequence reads and one or more off-target sequence reads based on the sequence read data. In certain embodiments, the one or more processors may then identify, based on the one or more off-target sequence reads, one or more intervals of off-target sequence read data corresponding to an enhancer of zest homologs inhibitory protein (EZHIP). In certain embodiments, the one or more processors may then modify the one or more on-target sequence reads to include the one or more intervals of off-target sequence read data corresponding to the EZHIP. In certain embodiments, the one or more processors may then perform a genomic variant call utilizing the modified one or more on-target sequence reads.

In certain embodiments, a genomic variant calling system may obtain at least one sample from an individual. In certain embodiments, the genomic variant calling system may isolate nucleic acids from the at least one sample. In certain embodiments, the genomic variant calling system sequence the isolated nucleic acids to produce a plurality of sequence reads. In certain embodiments, one or more processors of the genomic variant calling system may receive sequence read data based on the plurality of sequence reads. In certain embodiments, the one or more processors may then determine one or more on-target sequence reads and one or more off-target sequence reads based on the sequence read data. In certain embodiments, the one or more processors may identify one or more intervals of off-target sequence read data based on the one or more off-target sequence reads. In certain embodiments, the one or more processors may then determine one or more off-target sequence reads to be included in the one or more on-target sequence reads. In certain embodiments, the one or more processors may modify the one or more on-target sequence reads to include the one or more intervals of sequence read data associated with the off-target sequence reads determined to be included in the on-target sequence reads. In certain embodiments, the one or more processors may then perform a genomic variant call utilizing the modified one or more on-target sequence reads.

In certain embodiments, a genomic variant calling system may provide a plurality of nucleic acid molecules obtained from a sample. In certain embodiments, the genomic variant calling system may then ligate one or more adapters onto one or more nucleic acid molecules from the plurality of nucleic acid molecules. In certain embodiments, the genomic variant calling system may amplify the one or more ligated nucleic acid molecules from the plurality of nucleic acid molecules. In certain embodiments, the genomic variant calling system may capture amplified nucleic acid molecules from the amplified nucleic acid molecules. In certain embodiments, the genomic variant calling system may then sequence, by a sequencer, the captured nucleic acid molecules to obtain a plurality of sequence reads that represent a set of subgenomic intervals in the nucleic acid molecules. In certain embodiments, one or more processors of the genomic variant calling system may receive sequence read data based on the plurality of sequence reads. In certain embodiments, the one or more processors may determine one or more on-target sequence reads and one or more off-target sequence reads based on the sequence read data. In certain embodiments, the one or more processors may then identify one or more intervals of off-target sequence read data based on the one or more off-target sequence reads. In certain embodiments, the one or more processors may determine one or more off-target sequence reads to be included in the one or more on-target sequence reads. In certain embodiments, the one or more processors may modify the one or more on-target sequence reads to include the one or more intervals of sequence read data associated with the off-target sequence reads determined to be included in the on-target sequence reads. In certain embodiments, the one or more processors may perform a genomic variant call utilizing the modified one or more on-target sequence reads.

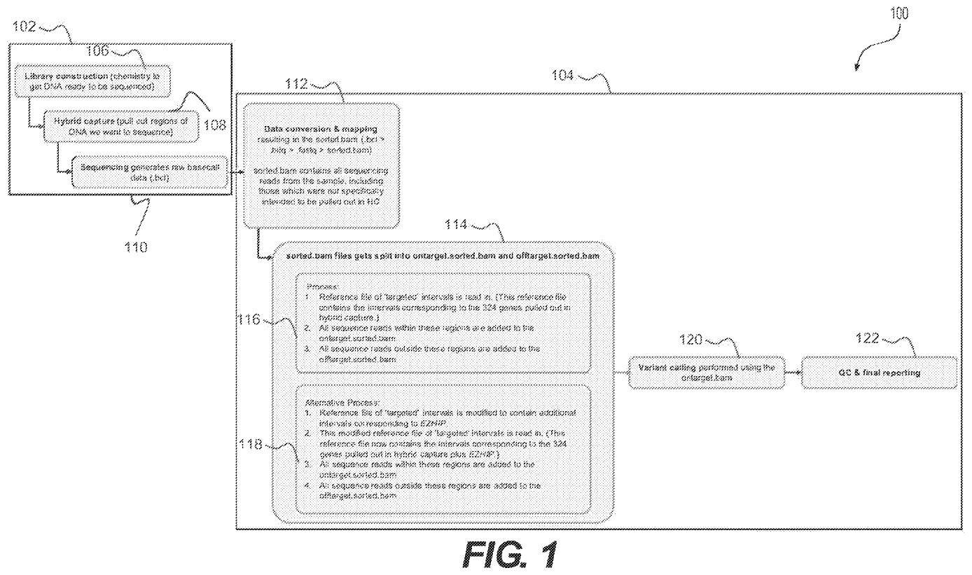

FIG. 1 illustrates a genomic variant calling system 100 for identifying one or more off-target sequence reads of interest and performing genomic variant calls based on the identified off-target sequence reads, in accordance with the disclosed embodiments. For example, in some embodiments, in the central nervous system (CNS), the loss of H3 lysine 27 hypo-trimethylation (H3K27me3) expression may, in some embodiments, include a hallmark of two different tumor types: diffuse midline glioma (DMG), H3K27-mutant and posterior fossa ependymoma, group PFA (PFA-EPN). In certain embodiments, Enhancer of Zest Homologs Inhibitory Protein (EZHIP) may be overexpressed (e.g., due to gene overexpression rather than mutations of the CXorf67 gene) in the large majority of PFA-EPN, and in the remaining cases of DMG showing H3K27me3 loss but lacking histone gene (H3) mutations. Indeed, both H3K27M and EZHIP may include competitive inhibitors of Polycomb Repressive Complex 2 (PRC2 ), which may interact with unmethylated CpG islands and is involved with transcriptional silencing. Thus, in accordance with the presently disclosed embodiments, it may be useful to capture both H3K27M and EZHIP by identifying one or more off-target sequence reads of interest (e.g., off-target sequence reads corresponding to EZHIP, H3K27M, or other similar off-target expression or mutation) and then performing genomic variant calls based on the identified off-target sequence reads. Specifically, in accordance with the presently disclosed embodiments, one or more reference files including on-target sequence reads may be modified to include off-target sequence reads corresponding, in one example, to EZHIP, and thus genomic variant calls of the modified one or more reference files may include genomic variant calls of the on-target sequence reads and the off-target sequence reads (e.g., corresponding to EZHIP, H3K27M, or other similar off-target expression or mutation).

In certain embodiments, the genomic variant calling system 100 may include a laboratory process and workflow 102 and a computing workflow 104. In certain embodiments, the laboratory process and workflow 102 may include a laboratory subprocess 106, in which a DNA sequence may be prepared to be sequenced. For example, in some embodiments, a sample from a subject may be obtained and DNA may be isolated from the sample. In certain embodiments, the laboratory process and workflow 102 may further include a hybrid capture subprocess 108, in which, for example, one or more sequence reads may be extracted from the DNA sequence. In certain embodiments, the laboratory process and workflow 102 may further include a sequencing subprocess 110, in which, for example, isolated DNA may be sequenced to produce a number of sequence reads. In one embodiment, the sequencing subprocess 108 may generate raw base call data as a Binary Base Call (BCL) file (e.g., “.bcl”).

In certain embodiments, the computing workflow 104 may begin with data conversion and mapping block 112. For example, in some embodiments, the data conversion and mapping block 112 may be performed to convert and map the raw base call data of the BCL file into a sorted binary alignment map (BAM) file (e.g., “.bcl”>“.bifq”>“.fastq”>“sorted.bam”). In some embodiments the sorted BAM file may include, for example, all sequencing reads extracted from the sample, including those which were not specifically intended to be extracted by the hybrid capture subprocess 108. In certain embodiments, the computing workflow 104 may continue with on-target and off-target sequence block 114 in which, for example, a reference file of one or more intervals corresponding to on-target sequences may be read (illustrated process 116). For example, in one embodiment, the reference file may include, for example, the intervals of the sequence corresponding to 324 genes intended to be extracted by the hybrid capture subprocess 108. In certain embodiments, all sequence reads within these intervals may be added to an ontarget.sorted.bam file, and all sequence reads outside these intervals may be added to an offtarget.sorted.bam file.

In certain embodiments, in accordance with the presently disclosed embodiments, the ontarget.sorted.bam file including on-target sequence reads may be modified to include off-target sequence reads corresponding, in one example, to EZHIP (alternative process 118). For example, in certain embodiments, the ontarget.sorted.bam file may be modified to include both the intervals of the sequence corresponding to 324 genes and intervals of the sequence corresponding to EZHIP. In certain embodiments, all sequence reads within these intervals may be added to the ontarget.sorted.bam file, and all sequence reads outside these intervals may be added to an offtarget.sorted.bam file. In certain embodiments, the computing workflow 104 may continue with genomic variant calling block 120 in which, for example, one or more genomic variant calls may be performed utilizing the modified ontarget.sorted.bam file including both the intervals of the sequence corresponding to 324 genes and intervals of the sequence corresponding to EZHIP. In certain embodiments, the computing workflow 104 may continue with quality control and reporting block 122 in which, for example, a quality control process based on the one or more genomic variant calls may be performed and a final report may be generated based on the one or more genomic variant calls.

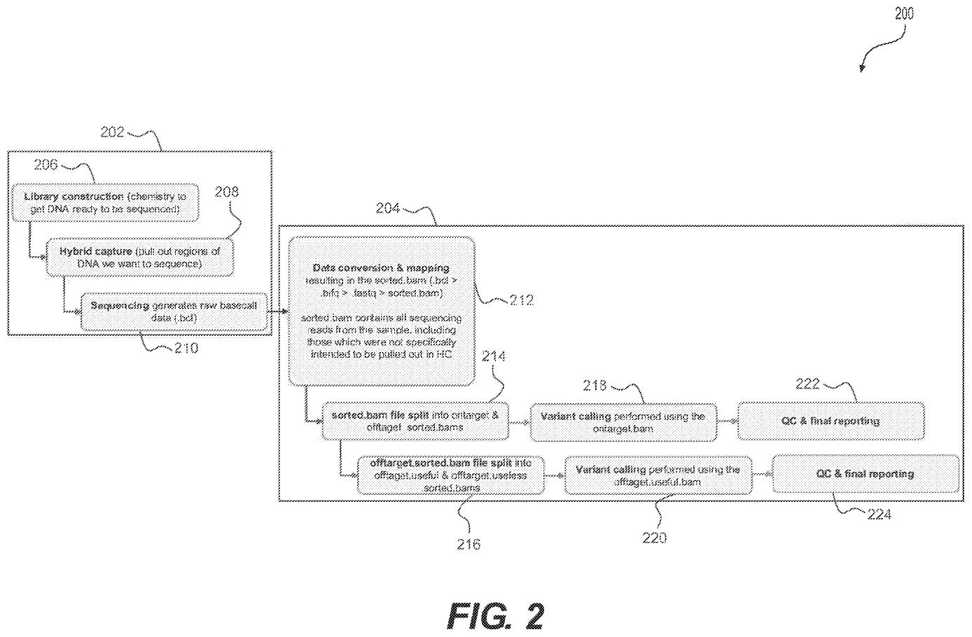

FIG. 2 illustrates a genomic variant calling system 100 for identifying one or more off-target sequence reads of interest and performing genomic variant calls based on the identified off-target sequence reads, in accordance with the disclosed embodiments. In certain embodiments, the genomic variant calling system 200 may include a laboratory process and workflow 202 and a computing workflow 204. In certain embodiments, the laboratory process and workflow 202 may include a laboratory subprocess 206, in which a DNA sequence may be prepared to be sequenced. For example, in some embodiments, a sample from a subject may be obtained and DNA may be isolated from the sample. In certain embodiments, the laboratory process and workflow 202 may further include a hybrid capture subprocess 208, in which, for example, one or more sequence reads may be extracted from the DNA sequence. In certain embodiments, the laboratory process and workflow 202 may further include a sequencing subprocess 210, in which, for example, isolated DNA may be sequenced to produce a number of sequence reads. In one embodiment, the sequencing subprocess 108 may generate raw base call data as a BCL file (e.g., “.bcl”).

In certain embodiments, the computing workflow 204 may begin with data conversion and mapping block 212. For example, in some embodiments, the data conversion and mapping block 212 may be performed to convert and map the raw base call data of the BCL file into a sorted binary alignment map (BAM) file (e.g., “.bcl”>“.bifq”>“.fastq”>“sorted.bam”). In some embodiments the sorted BAM file may include, for example, all sequencing reads extracted from the sample, including those which were not specifically intended to be extracted by the hybrid capture subprocess 208. In certain embodiments, the computing workflow 204 may continue with on-target block 214 in which, for example, a reference file of one or more intervals corresponding to on-target sequences may be read. For example, in one embodiment, the reference file may include, for example, the intervals of the sequence corresponding to 324 genes intended to be extracted by the hybrid capture subprocess 208. In certain embodiments, the computing workflow 204 may continue with on-target block 216 in which, for example, a reference file of one or more intervals corresponding to off-target sequences may be read. For example, in one embodiment, the reference file may include, for example, intervals of the sequence corresponding to EZHIP. In certain embodiments, all sequence reads within the intervals of the sequence corresponding to 324 genes may be added to an ontarget.sorted.bam file, and all sequence reads outside these intervals and the intervals of the sequence corresponding to EZHIP may be added to an offtarget.sorted.bam file.

In certain embodiments, the computing workflow 204 may continue with respective genomic variant calling blocks 218 and 220 in which, for example, one or more genomic variant calls may be performed utilizing the modified ontarget.sorted.bam file including the intervals of the sequence corresponding to 324 genes and utilizing the modified offtarget.sorted.bam file including intervals of the sequence corresponding to EZHIP, respectively. In certain embodiments, the computing workflow 204 may continue with respective quality control and reporting blocks 222 and 224 in which, for example, a quality control process based on the one or more genomic variant calls may be performed and a final report may be generated based on the one or more genomic variant calls, respectively.

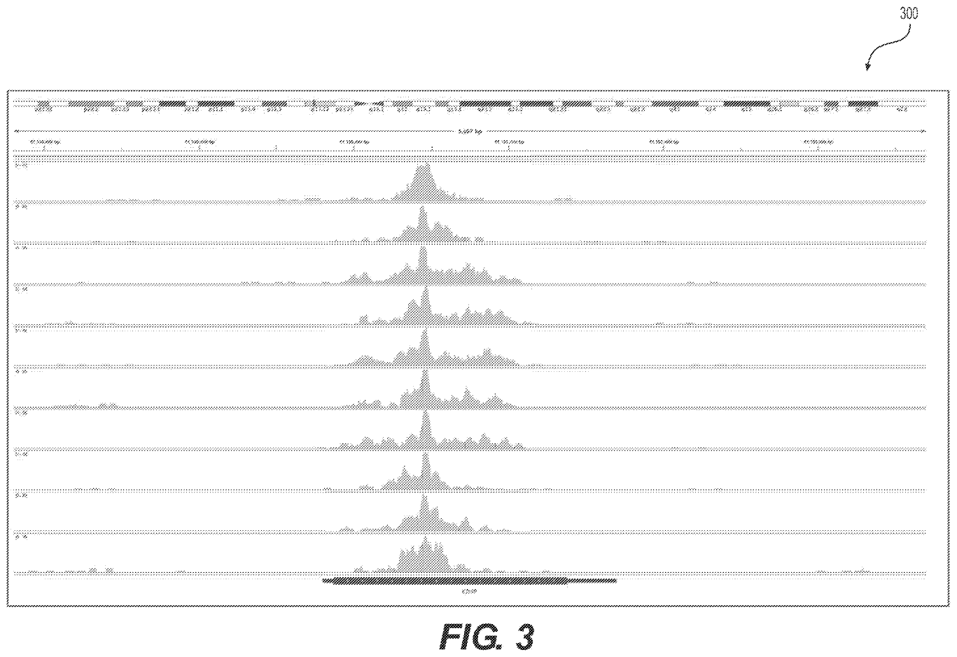

FIG. 3 illustrates an exemplary diagram 300 of one or more example on-target sequence reads and off-target sequence reads, in accordance with the disclosed embodiments As depicted, an interval of an on-target sequence read, for example, including the 324 genes may be captured and intervals of an off-target sequence read (e.g., outside of the bounds of the interval of an on-target sequence read) of the sequence corresponding to EZHIP may be captured in accordance with the presently disclosed embodiments.

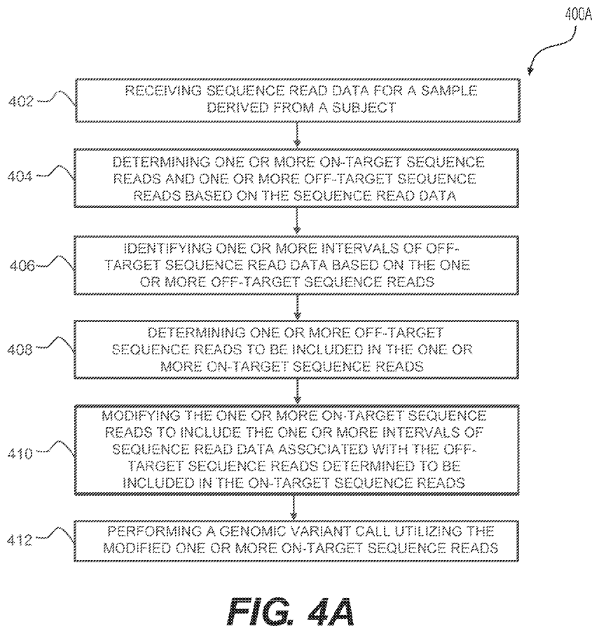

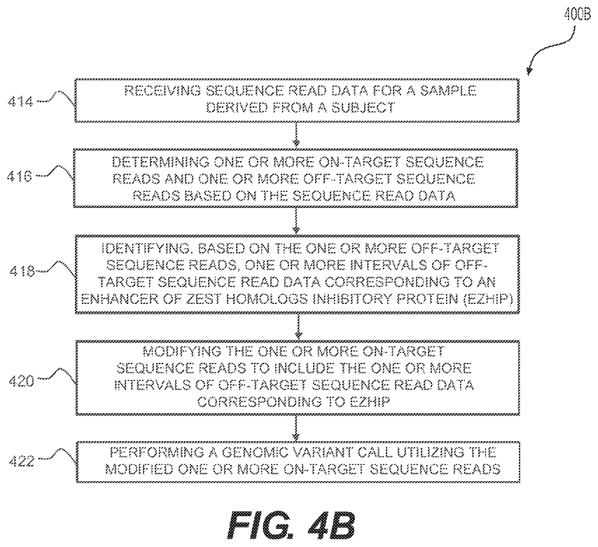

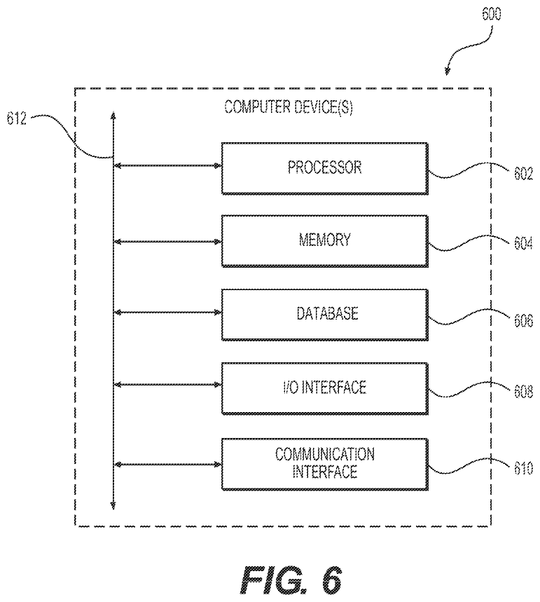

FIG. 4A illustrates a flow diagram of a method 400A for identifying one or more off-target sequence reads of interest and performing genomic variant calls based on the identified off-target sequence reads, in accordance with the disclosed embodiments. The method 400A may be performed utilizing one or more processing devices (e.g., computing device(s) to be discussed below with respect to FIG. 6) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for instructions running/executing on one or more processors), firmware (e.g., microcode), or some combination thereof.

The method 400A may begin at block 402 with the one or more processing devices receiving sequence read data for a sample derived from a subject. The method 400A may then continue at block 404 with the one or more processing devices determining one or more on-target sequence reads and one or more off-target sequence reads based on the sequence read data. The method 400A may then continue at block 406 with identifying one or more intervals of off-target sequence read data based on the one or more off-target sequence reads. The method 400A may then continue at block 408 with one or more processing devices determining one or more off-target sequence reads to be included in the one or more on-target sequence reads. The method 400A may then continue at block 410 with one or more processing devices modifying the one or more on-target sequence reads to include the one or more intervals of sequence read data associated with the off-target sequence reads determined to be included in the on-target sequence reads. The method 400A may then conclude at block 412 with one or more processing devices performing, using the one or more processors, a genomic variant call utilizing the modified one or more on-target sequence reads.

FIG. 4B illustrates a flow diagram of a method 400B for identifying one or more off-target sequence reads corresponding to EZHIP and performing genomic variant calls based on the identified off-target sequence reads, in accordance with the disclosed embodiments. The method 400B may be performed utilizing one or more processing devices (e.g., computing device(s) to be discussed below with respect to FIG. 6) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for instructions running/executing on one or more processors), firmware (e.g., microcode), or some combination thereof.

The method 400B may begin at block 414 with the one or more processing devices receiving sequence read data for a sample derived from a subject. The method 400B may then continue at block 416 with the one or more processing devices determining one or more on-target sequence reads and one or more off-target sequence reads based on the sequence read data. The method 400B may then continue at block 418 with identifying, based on the one or more off-target sequence reads, one or more intervals of off-target sequence read data corresponding to an enhancer of zest homologs inhibitory protein (EZHIP). The method 400B may then continue at block 420 with one or more processing devices modifying the one or more on-target sequence reads to include the one or more intervals of off-target sequence read data corresponding to the EZHIP. The method 400B may then conclude at block 422 with one or more processing devices performing a genomic variant call utilizing the modified one or more on-target sequence reads.

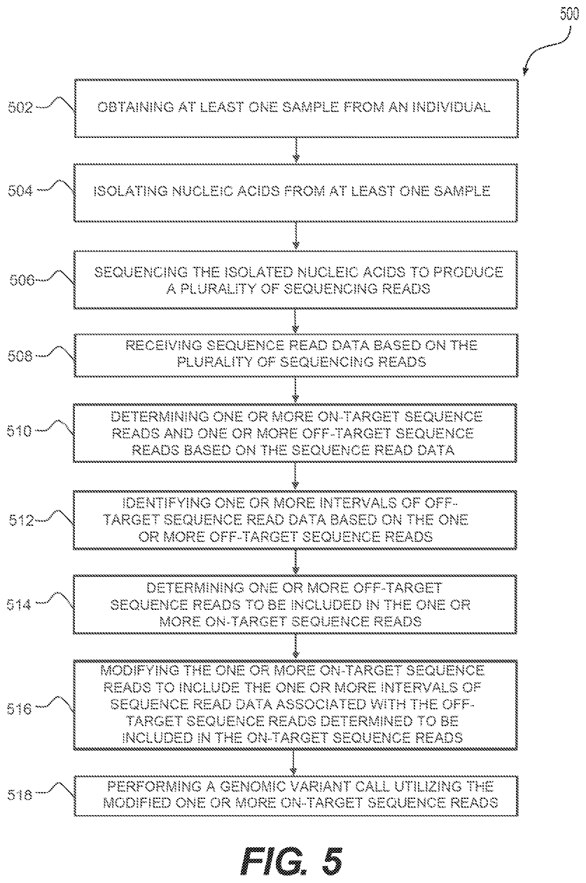

FIG. 5 illustrates a flow diagram of a method 500 for identifying one or more off-target sequence reads of interest and performing genomic variant calls based on the identified off-target sequence reads, in accordance with the disclosed embodiments. The method 500 may be performed utilizing one or more processing devices (e.g., computing device(s) to be discussed below with respect to FIG. 6) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various omics data and making one or more decisions based thereon), software (e.g., instructions running/executing on one or more processors), firmware (e.g., microcode), or some combination thereof.

The method 500 may begin at block 502 with obtaining at least one sample from an individual. The method 500 may then continue at block 504 with isolating nucleic acids from the at least one sample. The method 500 may then continue at block 506 with sequencing the isolated nucleic acids to produce a plurality of sequence reads. The method 500 may then continue at block 508 with one or more processing devices receiving sequence read data based on the plurality of sequence reads. The method 500 may then continue at block 510 with one or more processing devices determining one or more on-target sequence reads and one or more off-target sequence reads based on the sequence read data. The method 500 may then continue at block 512 with one or more processing devices identifying one or more intervals of off-target sequence read data based on the one or more off-target sequence reads. The method 500 may then continue at block 514 with one or more processing devices determining one or more off-target sequence reads to be included in the one or more on-target sequence reads. The method 500 may then continue at block 516 with one or more processing devices modifying the one or more on-target sequence reads to include the one or more intervals of sequence read data associated with the off-target sequence reads determined to be included in the on-target sequence reads. The method 500 may then continue at block 516 with one or more processing devices performing a genomic variant call utilizing the modified one or more on-target sequence reads.

In some instances, the gene panel may comprise at least 1, at least 2, at least 3, at least 4, at least 5, at least 6, at least 7, at least 8, at least 9, at least 10, at least 20, at least 30, at least 40, or more than 40 genes.

In some instances, the disclosed methods may be used to generate a report of genomic and medical information associated with a patient by assessing genomic and medical information in at least 1, at least 2, at least 3, at least 4, at least 5, at least 6, at least 7, at least 8, at least 9, at least 10, at least 20, at least 30, at least 40, or more than 40 gene loci.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for identifying off-target sequence reads of interest and performing genomic variant calls based on the identified off-target sequence reads may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for identifying off-target sequence reads of interest and performing genomic variant calls based on the identified off-target sequence reads may be used to select a subject (e.g., a patient) for a clinical trial based on the clinically significant genomic and medical information value determined for one or more gene loci. In some instances, patient selection for clinical trials based on, e.g., identification of clinically significant genomic and medical information at one or more gene loci, may accelerate the development of targeted therapies and improve the healthcare outcomes for treatment decisions.

In some instances, the disclosed methods for identifying off-target sequence reads of interest and performing genomic variant calls based on the identified off-target sequence reads may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for identifying off-target sequence reads of interest and performing genomic variant calls based on the identified off-target sequence reads may be used in treating a disease (e.g., a cancer) in a subject. For example, in response to determining clinically significant genomic and medical information using any of the methods disclosed herein, an effective amount of an anti-cancer therapy or anti-cancer treatment may be administered to the subject.

In some instances, the disclosed methods for identifying off-target sequence reads of interest and performing genomic variant calls based on the identified off-target sequence reads may be used for monitoring disease progression or recurrence (e.g., cancer or tumor progression or recurrence) in a subject. For example, in some instances, the methods may be used to determine clinically significant genomic and medical information in a first sample obtained from the subject at a first time point, and used to determine clinically significant genomic and medical information in a second sample obtained from the subject at a second time point, where comparison of the first determination of clinically significant genomic and medical information and the second determination of clinically significant genomic and medical information allows one to monitor disease progression or recurrence. In some instances, the first time point is chosen before the subject has been administered a therapy or treatment, and the second time point is chosen after the subject has been administered the therapy or treatment.

In some instances, the disclosed methods for identifying off-target sequence reads of interest and performing genomic variant calls based on the identified off-target sequence reads may be used for adjusting a therapy or treatment (e.g., an anti-cancer treatment or anti-cancer therapy) for a subject, e.g., by adjusting a treatment dose and/or selecting a different treatment in response to a change in the determination of clinically significant genomic and medical information.

In some instances, the value of clinically significant genomic and medical information determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for identifying off-target sequence reads of interest and performing genomic variant calls based on the identified off-target sequence reads may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for generating a report of genomic and medical information associated with a patient as part of a genomic profiling process (or inclusion of the output from the disclosed methods for generating a report of genomic and medical information associated with a patient as part of the genomic profile of the subject) can improve the validity of, e.g., disease detection calls and treatment decisions, made on the basis of the genomic profile by, for example, independently confirming the presence of clinically significant genomic and medical information in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM 333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB 351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D 3399-00, D 3399-01, and D 3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 400, or 500 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or nonspecific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500,greater than 600, greater than 700, greater than 800, greater than 400, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 500 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 400 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 500 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 400 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing,” and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 400, at least 450, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1470) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5):589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e−6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6):730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 500, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 400, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for identifying off-target sequence reads of interest and performing genomic variant calls based on the identified off-target sequence reads. The systems may comprise, e.g., one or more processors, and a memory unit communicatively coupled to the one or more processors and configured to store instructions that, when executed by the one or more processors, cause the system perform operations including receiving, at the one or more processors, genomic testing data associated with the patient; based on the genomic testing data, retrieving, at the one or more processors, medical information including one or more potential clinical treatments for the patient; determining, by the one or more processors, that the medical information has at least some clinical significance to the patient; based on at least a portion of the medical information having at least some clinical significance to the patient, generating, by the one or more processors, patient-specific medical data; determining, by the one or more processors, at least one specific position to dispose the medical information on the report based on the patient-specific medical data; and generating, by the one or more processors, the report based on the determined specific position.

In some instances, the disclosed systems may further comprise a sequencer, e.g., a next generation sequencer (also referred to as a massively parallel sequencer). Examples of next generation (or massively parallel) sequencing platforms include, but are not limited to, the Roche 454, Illumina Solexa, ABI-SOLiD, ION Torrent, or Pacific Bioscience sequencing platforms.

In some instances, the disclosed systems may be used for generating a report of genomic and medical information associated with a patient in any of a variety of samples as described herein (e.g., a tissue sample, biopsy sample, hematological sample, or liquid biopsy sample derived from the subject).

In some instances, the plurality of gene loci for which sequencing data is processed to determine clinically significant genomic and medical information may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of clinically significant genomic and medical information is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

FIG. 4 illustrates an example of a computing device or system in accordance with one embodiment.

FIG. 6 illustrates an example of one or more computing device(s) 600 that may be utilized for identifying off-target sequence reads of interest and performing genomic variant calls based on the identified off-target sequence reads, in accordance with the disclosed embodiments. In certain embodiments, the one or more computing device(s) 600 may perform one or more steps of one or more methods described or illustrated herein. In certain embodiments, the one or more computing device(s) 600 provide functionality described or illustrated herein. In certain embodiments, software running on the one or more computing device(s) 600 performs one or more steps of one or more methods described or illustrated herein, or provides functionality described or illustrated herein. Certain embodiments include one or more portions of the one or more computing device(s) 600.

This disclosure contemplates any suitable number of computing systems 600. This disclosure contemplates one or more computing device(s) 600 taking any suitable physical form. As example and not by way of limitation, one or more computing device(s) 600 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 600 may be unitary or distributed; span multiple locations; span multiple machines; span multiple data centers; or reside in a cloud, which may include one or more cloud components in one or more networks.

Where appropriate, the one or more computing device(s) 600 may perform without substantial spatial or temporal limitation one or more steps of one or more methods described or illustrated herein. As an example, and not by way of limitation, the one or more computing device(s) 600 may perform, in real-time or in batch mode, one or more steps of one or more methods described or illustrated herein. The one or more computing device(s) 600 may perform, at different times or at different locations, one or more steps of one or more methods described or illustrated herein, where appropriate.

In certain embodiments, the one or more computing device(s) 600 includes a processor 602, memory 604, database 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement. In certain embodiments, processor 602 includes hardware for executing instructions, such as those making up a computer program. As an example, and not by way of limitation, to execute instructions, processor 602 may retrieve (or fetch) the instructions from an internal register, an internal cache, memory 604, or database 606; decode and execute them; and then write one or more results to an internal register, an internal cache, memory 604, or database 606. In certain embodiments, processor 602 may include one or more internal caches for data, instructions, or addresses. This disclosure contemplates processor 602 including any suitable number of any suitable internal caches, where appropriate. As an example, and not by way of limitation, processor 602 may include one or more instruction caches, one or more data caches, and one or more translation lookaside buffers (TLBs). Instructions in the instruction caches may be copies of instructions in memory 604 or database 606, and the instruction caches may speed up retrieval of those instructions by processor 602.

Data in the data caches may be copies of data in memory 604 or database 606 for instructions executing at processor 602 to operate on; the results of previous instructions executed at processor 602 for access by subsequent instructions executing at processor 602 or for writing to memory 604 or database 606; or other suitable data. The data caches may speed up read or write operations by processor 602. The TLBs may speed up virtual-address translation for processor 602. In certain embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In certain embodiments, memory 604 includes main memory for storing instructions for processor 602 to execute or data for processor 602 to operate on. As an example, and not by way of limitation, the one or more computing device(s) 600 may load instructions from database 606 or another source (such as, for example, another one or more computing device(s) 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604.

In certain embodiments, processor 602 executes only instructions in one or more internal registers, internal caches, or memory 604 (as opposed to database 606 or elsewhere) and operates only on data in one or more internal registers, internal caches, or memory 604 (as opposed to database 606 or elsewhere). One or more memory buses (which may each include an address bus and a data bus) may couple processor 602 to memory 604. Bus 612 may include one or more memory buses, as described below. In certain embodiments, one or more memory management units (MMUs) reside between processor 602 and memory 604 and facilitate accesses to memory 604 requested by processor 602. In certain embodiments, memory 604 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memory devices 604, where appropriate. Although this disclosure describes and illustrates particular memory, this disclosure contemplates any suitable memory.

In certain embodiments, database 606 includes mass storage for data or instructions. As an example, and not by way of limitation, database 606 may include a hard disk drive (HDD), a floppy disk drive, flash memory, an optical disc, a magneto-optical disc, magnetic tape, or a Universal Serial Bus (USB) drive, or a combination of two or more of these. Database 606 may include removable or non-removable (or fixed) media, where appropriate. Database 606 may be internal or external to the one or more computing device(s) 600, where appropriate. In certain embodiments, database 606 is non-volatile, solid-state memory. In certain embodiments, database 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), flash memory, or a combination of two or more of these. This disclosure contemplates mass database 606 taking any suitable physical form. Database 606 may include one or more storage control units facilitating communication between processor 602 and database 606, where appropriate. Where appropriate, database 606 may include one or more databases 606. Although this disclosure describes and illustrates particular storage, this disclosure contemplates any suitable storage.

In certain embodiments, I/O interface 608 includes hardware, software, or both, providing one or more interfaces for communication between the one or more computing device(s) 600 and one or more I/O devices. The one or more computing device(s) 600 may include one or more of these I/O devices, where appropriate. One or more of these I/O devices may enable communication between a person and the one or more computing device(s) 600. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In certain embodiments, communication interface 610 includes hardware, software, or both providing one or more interfaces for communication (such as, for example, packet-based communication) between the one or more computing device(s) 600 and one or more other computing device(s) 600 or one or more networks. As an example, and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it.

As an example, and not by way of limitation, the one or more computing device(s) 600 may communicate with an ad hoc network, a personal area network (PAN), a local area network (LAN), a wide area network (WAN), a metropolitan area network (MAN), one or more portions of the Internet, or a combination of two or more of these. One or more portions of one or more of these networks may be wired or wireless. As an example, the one or more computing device(s) 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), other suitable wireless network, or a combination of two or more of these. The one or more computing device(s) 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In certain embodiments, bus 612 includes hardware, software, or both coupling components of the one or more computing device(s) 600 to each other. As an example, and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, another suitable bus, or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

### Exemplary Implementations

Exemplary implementations of the methods and systems described herein include:

1. A method, comprising:
receiving, at one or more processors, sequence read data for a sample derived from a subject;
determining, using the one or more processors, one or more on-target sequence reads and one or more off-target sequence reads based on the sequence read data;
identifying, using the one or more processors, one or more intervals of off-target sequence read data based on the one or more off-target sequence reads;
determining, using the one or more processors, one or more off-target sequence reads to be included in the one or more on-target sequence reads;
modifying, using the one or more processors, the one or more on-target sequence reads to include the one or more intervals of sequence read data associated with the off-target sequence reads determined to be included in the on-target sequence reads; and
performing, using the one or more processors, a genomic variant call utilizing the modified one or more on-target sequence reads.

2. The method of clause 1, further comprising performing, using the one or more processors, a quality control process based on the genomic variant call.

3. The method of any of clauses 1-2, further comprising:
generating, using the one or more processors, a report based on the genomic variant call.

4. The method of clause 1, further comprising:
prior to receiving the sequence read data:
   performing a hybridization-capture process to extract genomic reads for next-generation sequencing (NGS); and
   performing the NGS to generate a plurality of base-call sequencing data based on the extracted genomic reads.

5. The method of clause 4, further comprising converting the plurality of base-call sequencing data into a sorted binary alignment map (BAM) file.

6. The method of clause 1, wherein receiving the sequence read data comprises receiving, using the one or more processors, sequence reads corresponding to a targeted 324 genes.

7. The method of clause 6, further comprising generating, using the one or more processors, a reference file of intervals of sequence read data based on the targeted 324 genes and the one or more intervals of off-target sequence read data.

8. The method of clause 7, further comprising performing, using the one or more processors, a genomic variant call utilizing the reference file.

9. The method of clause 1, further comprising:
prior to modifying the one or more on-target sequence reads to include the one or more intervals of off-target sequence read data:
   performing, using the one or more processors, a genomic variant call utilizing the one or more on-target sequence reads.

10. The method of clause 9, further comprising:
prior to modifying the one or more on-target sequence reads to include the one or more intervals of off-target sequence read data:
   performing, using the one or more processors, a genomic variant call utilizing the one or more off-target sequence reads, the genomic variant call corresponding to the one or more intervals of off-target sequence read data.

11. The method of clause 1, wherein identifying the one or more intervals of off-target sequence read data comprises identifying one or more base-pair (bp) intervals outside of a predetermined bp interval.

12. The method of clause 1, wherein performing the genomic variant call utilizing the modified one or more on-target sequence reads comprises identifying an indication of a genetic biomarker of the sample.

13. The method of clause 12, wherein the genetic biomarker of the sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

14. The method of any of clauses 12-13, wherein the sample comprises a tumor.

15. The method of any of clauses 12-14, wherein the tumor comprises at least one of a diffuse midline glioma (DMG), H3K27-mutant tumor, a posterior fossa ependymoma group PFA (PFA-EFN) tumor, a DMG, H3K27-WT tumor with enhancer of zest homologs inhibitory protein (EZHIP) overexpression, a wingless-activated (WNT) medulloblastoma tumor, an atypical teratoid/rhabdoid tumor (AT/RT), or a germinoma tumor.

16. The method of clause 1, further comprising identifying, using the one or more processors, one or more intervals of off-target sequence read data corresponding to one or more of an enhancer of zest homologs inhibitory protein (EZHIP), H3 lysine 27 hypo-trimethylation (H3K27me3), or HIST1H3B K27M.

17. The method of clause 1, wherein performing the genomic variant call comprises performing, using the one or more processors, a genotypic or phenotypic call utilizing the modified one or more on-target sequence reads.

18. The method of clause 1, wherein the off-target sequence reads comprise less than 50%, less than 40%, less than 30%, less than 20%, or less than 10% of the sequence reads in the sequence read data.

19. The method of clause 1, further comprising causing one or more electronic devices to display a report generated based on the genomic variant call.

20. The method of clause 19, wherein causing the one or more electronic devices to display the report comprises causing a human machine interface (HMI) associated with a pathologist or a clinician to display the report.

21. A system including one or more computing devices, comprising:
one or more non-transitory computer-readable storage media including instructions; and
one or more processors coupled to the one or more storage media, the one or more processors configured to execute the instructions to:
   receive sequence read data for a sample derived from a subject;
   determine one or more on-target sequence reads and one or more off-target sequence reads based on the sequence read data;
   identify one or more intervals of off-target sequence read data based on the one or more off-target sequence reads;
   determine one or more off-target sequence reads to be included in the one or more on-target sequence reads;
   modify the one or more on-target sequence reads to include the one or more intervals of sequence read data associated with the off-target sequence reads determined to be included in the on-target sequence reads; and
   perform a genomic variant call utilizing the modified one or more on-target sequence reads.

22. The system of clause 21, wherein the instructions further comprise instructions to comprising perform a quality control process based on the genomic variant call.

23. The system of any of clauses 21-22, wherein the instructions further comprise instructions to:
generate a report based on the genomic variant call.

24. The system of clause 21, wherein the instructions further comprise instructions to:
prior to receiving the sequence read data:
   perform a hybridization-capture process to extract genomic reads for next-generation sequencing (NGS); and
   perform the NGS to generate a plurality of base-call sequencing data based on the extracted genomic reads.

25. The system of clause 24, wherein the instructions further comprise instructions to convert the plurality of base-call sequencing data into a sorted binary alignment map (BAM) file.

26. The system of clause 21, wherein the instructions to receive the sequence read data further comprise instructions to receive sequence reads corresponding to a targeted 324 genes.

27. The system of clause 26, wherein the instructions further comprise instructions to generate a reference file of intervals of sequence read data based on the targeted 324 genes and the one or more intervals of off-target sequence read data.

28. The system of clause 27, wherein the instructions further comprise instructions to perform a genomic variant call utilizing the reference file.

29. The system of clause 21, wherein the instructions further comprise instructions to:
prior to modifying the one or more on-target sequence reads to include the one or more intervals of off-target sequence read data:
   perform a genomic variant call utilizing the one or more on-target sequence reads.

30. The system of clause 29, wherein the instructions further comprise instructions to:
prior to modifying the one or more on-target sequence reads to include the one or more intervals of off-target sequence read data:
   perform a genomic variant call utilizing the one or more off-target sequence reads, the genomic variant call corresponding to the one or more intervals of off-target sequence read data.

31. The system of clause 21, wherein the instructions to identify the one or more intervals of off-target sequence read data further comprise instructions to identify one or more base-pair (bp) intervals outside of a predetermined bp interval.

32. The system of clause 21, wherein the instructions to perform the genomic variant call utilizing the modified one or more on-target sequence reads further comprise instructions to identify an indication of a genetic biomarker of the sample.

33. The system of clause 32, wherein the genetic biomarker of the sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

34. The system of any of clauses 32-33, wherein the sample comprises a tumor.

35. The system of any of clauses 32-34, wherein the tumor comprises at least one of a diffuse midline glioma (DMG), H3K27-mutant tumor, a posterior fossa ependymoma group PFA (PFA-EFN) tumor, a DMG, H3K27-WT tumor with enhancer of zest homologs inhibitory protein (EZHIP) overexpression, a wingless-activated (WNT) medulloblastoma tumor, an atypical teratoid/rhabdoid tumor (AT/RT), or a germinoma tumor.

36. The system of clause 21, wherein the instructions further comprise instructions to identify one or more intervals of off-target sequence read data corresponding to one or more of an enhancer of zest homologs inhibitory protein (EZHIP), H3 lysine 27 hypo-trimethylation (H3K27me3), or HIST1H3B K27M.

37. The system of clause 21, wherein the instructions to perform the genomic variant call further comprise instructions to perform a genotypic or phenotypic call utilizing the modified one or more on-target sequence reads.

38. The system of clause 21, wherein the off-target sequence reads comprise less than 50%, less than 40%, less than 30%, less than 20%, or less than 10% of the sequence reads in the sequence read data.

39. The system of clause 21, wherein the instructions further comprise instructions to cause one or more electronic devices to display a report generated based on the genomic variant call.

40. The system of clause 39, wherein the instructions to cause the one or more electronic devices to display the report further comprise instructions to cause a human machine interface (HMI) associated with a pathologist or a clinician to display the report.

41. A non-transitory computer-readable medium comprising instructions that, when executed by one or more processors of one or more computing devices, cause the one or more processors to:
receive sequence read data for a sample derived from a subject;
determine one or more on-target sequence reads and one or more off-target sequence reads based on the sequence read data;
identify one or more intervals of off-target sequence read data based on the one or more off-target sequence reads;
determine one or more off-target sequence reads to be included in the one or more on-target sequence reads;
modify the one or more on-target sequence reads to include the one or more intervals of sequence read data associated with the off-target sequence reads determined to be included in the on-target sequence reads; and
perform a genomic variant call utilizing the modified one or more on-target sequence reads.

42. The non-transitory computer-readable medium of clause 21, wherein the instructions further comprise instructions to comprising perform a quality control process based on the genomic variant call.

43. The non-transitory computer-readable medium of any of clauses 41-42, wherein the instructions further comprise instructions to:
generate a report based on the genomic variant call.

44. The non-transitory computer-readable medium of clause 41, wherein the instructions further comprise instructions to:
prior to receiving the sequence read data:
   perform a hybridization-capture process to extract genomic reads for next-generation sequencing (NGS); and
   perform the NGS to generate a plurality of base-call sequencing data based on the extracted genomic reads.

45. The non-transitory computer-readable medium of clause 44, wherein the instructions further comprise instructions to convert the plurality of base-call sequencing data into a sorted binary alignment map (BAM) file.

46. The non-transitory computer-readable medium of clause 41, wherein the instructions to receive the sequence read data further comprise instructions to receive sequence reads corresponding to a targeted 324 genes.

47. The non-transitory computer-readable medium of clause 46, wherein the instructions further comprise instructions to generate a reference file of intervals of sequence read data based on the targeted 324 genes and the one or more intervals of off-target sequence read data.

48. The non-transitory computer-readable medium of clause 47, wherein the instructions further comprise instructions to perform a genomic variant call utilizing the reference file.

49. The non-transitory computer-readable medium of clause 41, wherein the instructions further comprise instructions to:
prior to modifying the one or more on-target sequence reads to include the one or more intervals of off-target sequence read data:
   perform a genomic variant call utilizing the one or more on-target sequence reads.

50. The non-transitory computer-readable medium of clause 49, wherein the instructions further comprise instructions to:
prior to modifying the one or more on-target sequence reads to include the one or more intervals of off-target sequence read data:
   perform a genomic variant call utilizing the one or more off-target sequence reads, the genomic variant call corresponding to the one or more intervals of off-target sequence read data.

51. The non-transitory computer-readable medium of clause 41, wherein the instructions to identify the one or more intervals of off-target sequence read data further comprise instructions to identify one or more base-pair (bp) intervals outside of a predetermined bp interval.

52. The non-transitory computer-readable medium of clause 41, wherein the instructions to perform the genomic variant call utilizing the modified one or more on-target sequence reads further comprise instructions to identify an indication of a genetic biomarker of the sample.

53. The non-transitory computer-readable medium of clause 52, wherein the genetic biomarker of the sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

54. The non-transitory computer-readable medium of any of clauses 52-53, wherein the sample comprises a tumor.

55. The non-transitory computer-readable medium of any of clauses 52-54, wherein the tumor comprises at least one of a diffuse midline glioma (DMG), H3K27-mutant tumor, a posterior fossa ependymoma group PFA (PFA-EFN) tumor, a DMG, H3K27-WT tumor with enhancer of zest homologs inhibitory protein (EZHIP) overexpression, a wingless-activated (WNT) medulloblastoma tumor, an atypical teratoid/rhabdoid tumor (AT/RT), or a germinoma tumor.

56. The non-transitory computer-readable medium of clause 41, wherein the instructions further comprise instructions to identify one or more intervals of off-target sequence read data corresponding to one or more of an enhancer of zest homologs inhibitory protein (EZHIP), H3 lysine 27 hypo-trimethylation (H3K27me3), or HISTIH3B K27M.

57. The non-transitory computer-readable medium of clause 41, wherein the instructions to perform the genomic variant call further comprise instructions to perform a genotypic or phenotypic call utilizing the modified one or more on-target sequence reads.

58. The non-transitory computer-readable medium of clause 41, wherein the off-target sequence reads comprise less than 50%, less than 40%, less than 30%, less than 20%, or less than 10% of the sequence reads in the sequence read data.

59. The non-transitory computer-readable medium of clause 41, wherein the instructions further comprise instructions to cause one or more electronic devices to display a report generated based on the genomic variant call.

60. The non-transitory computer-readable medium of clause 59, wherein the instructions to cause the one or more electronic devices to display the report further comprise instructions to cause a human machine interface (HMI) associated with a pathologist or a clinician to display the report.

61. A method, comprising:
receiving, at one or more processors, sequence read data for a sample derived from a subject;
determining, using the one or more processors, one or more on-target sequence reads and one or more off-target sequence reads based on the sequence read data;
identifying, using the one or more processors, and based on the one or more off-target sequence reads, one or more intervals of off-target sequence read data corresponding to an enhancer of zest homologs inhibitory protein (EZHIP);
modifying, using the one or more processors, the one or more on-target sequence reads to include the one or more intervals of off-target sequence read data corresponding to the EZHIP; and
performing, using the one or more processors, a genomic variant call utilizing the modified one or more on-target sequence reads.

62. A method, comprising:
obtaining at least one sample from an individual;
isolating nucleic acids from the at least one sample;
sequencing the isolated nucleic acids to produce a plurality of sequence reads;
receiving, at one or more processors, sequence read data based on the plurality of sequence reads;
determining, using the one or more processors, one or more on-target sequence reads and one or more off-target sequence reads based on the sequence read data;
identifying, using the one or more processors, one or more intervals of off-target sequence read data based on the one or more off-target sequence reads;
determining, using the one or more processors, one or more off-target sequence reads to be included in the one or more on-target sequence reads;
modifying, using the one or more processors, the one or more on-target sequence reads to include the one or more intervals of sequence read data associated with the off-target sequence reads determined to be included in the on-target sequence reads; and
performing, using the one or more processors, a genomic variant call utilizing the modified one or more on-target sequence reads.

63. A method, comprising:
providing a plurality of nucleic acid molecules obtained from a sample;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent a set of subgenomic intervals in the nucleic acid molecules;
receiving, at one or more processors, sequence read data based on the plurality of sequence reads;
determining, using the one or more processors, one or more on-target sequence reads and one or more off-target sequence reads based on the sequence read data;
identifying, using the one or more processors, one or more intervals of off-target sequence read data based on the one or more off-target sequence reads;
determining, using the one or more processors, one or more off-target sequence reads to be included in the one or more on-target sequence reads;
modifying, using the one or more processors, the one or more on-target sequence reads to include the one or more intervals of sequence read data associated with the off-target sequence reads determined to be included in the on-target sequence reads; and performing, using the one or more processors, a genomic variant call utilizing the modified one or more on-target sequence reads.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method, comprising:
providing a plurality of nucleic acid molecules obtained from a sample;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent a set of subgenomic intervals in the nucleic acid molecules;
receiving, at one or more processors, sequence read data based on the plurality of sequence reads;
determining, using the one or more processors, one or more on-target sequence reads and one or more off-target sequence reads based on the sequence read data;
identifying, using the one or more processors, one or more intervals of off-target sequence read data based on the one or more off-target sequence reads;
determining, using the one or more processors, one or more off-target sequence reads to be included in the one or more on-target sequence reads;
modifying, using the one or more processors, the one or more on-target sequence reads to include the one or more intervals of sequence read data associated with the off-target sequence reads determined to be included in the on-target sequence reads; and
performing, using the one or more processors, a genomic variant call utilizing the modified one or more on-target sequence reads.

2. The method of claim 1, further comprising performing, using the one or more processors, a quality control process based on the genomic variant call.

3. The method of claim 1, further comprising:
generating, using the one or more processors, a report based on the genomic variant call.

4. The method of claim 1, further comprising:
prior to receiving the sequence read data:
performing a hybridization-capture process to extract genomic reads for next-generation sequencing (NGS); and
performing the NGS to generate a plurality of base-call sequencing data based on the extracted genomic reads.

5. The method of claim 4, further comprising converting the plurality of base-call sequencing data into a sorted binary alignment map (BAM) file.

6. The method of claim 1, wherein receiving the sequence read data comprises receiving, using the one or more processors, sequence reads corresponding to a targeted 324 genes.

7. The method of claim 6, further comprising generating, using the one or more processors, a reference file of intervals of sequence read data based on the targeted 324 genes and the one or more intervals of off-target sequence read data.

8. The method of claim 7, further comprising performing, using the one or more processors, a genomic variant call utilizing the reference file.

9. The method of claim 1, further comprising:
prior to modifying the one or more on-target sequence reads to include the one or more intervals of off-target sequence read data:
performing, using the one or more processors, a genomic variant call utilizing the one or more on-target sequence reads.

10. The method of claim 9, further comprising:
prior to modifying the one or more on-target sequence reads to include the one or more intervals of off-target sequence read data:
performing, using the one or more processors, a genomic variant call utilizing the one or more off-target sequence reads, the genomic variant call corresponding to the one or more intervals of off-target sequence read data.

11. The method of claim 1, wherein identifying the one or more intervals of off-target sequence read data comprises identifying one or more base-pair (bp) intervals outside of a predetermined bp interval.

12. The method of claim 1, wherein performing the genomic variant call utilizing the modified one or more on-target sequence reads comprises identifying an indication of a genetic biomarker of the sample.

13. The method of claim 12, wherein the genetic biomarker of the sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

14. The method of claim 12, wherein the sample comprises a tumor.

15. The method of claim 14, wherein the tumor comprises at least one of a diffuse midline glioma (DMG), H3K27-mutant tumor, a posterior fossa ependymoma group PFA (PFA-EFN) tumor, a DMG, H3K27-WT tumor with enhancer of zest homologs inhibitory protein (EZHIP) overexpression, a wingless-activated (WNT) medulloblastoma tumor, an atypical teratoid/rhabdoid tumor (AT/RT), or a germinoma tumor.

16. The method of claim 1, further comprising identifying, using the one or more processors, one or more intervals of off-target sequence read data corresponding to one or more of an enhancer of zest homologs inhibitory protein (EZHIP), H3 lysine 27 hypo-trimethylation (H3K27me3), or HIST1H3B K27M.

17. The method of claim 1, wherein performing the genomic variant call comprises performing, using the one or more processors, a genotypic or phenotypic call utilizing the modified one or more on-target sequence reads.

18. The method of claim 1, wherein the off-target sequence reads comprise less than 50%, less than 40%, less than 30%, less than 20%, or less than 10% of the sequence reads in the sequence read data.

19. (canceled)

20. (canceled)

21. A system including one or more computing devices, comprising:
one or more non-transitory computer-readable storage media including instructions; and
one or more processors coupled to the one or more storage media, the one or more processors configured to execute the instructions to:
receive sequence read data for a sample derived from a subject;
determine one or more on-target sequence reads and one or more off-target sequence reads based on the sequence read data;
identify one or more intervals of off-target sequence read data based on the one or more off-target sequence reads;
determine one or more off-target sequence reads to be included in the one or more on-target sequence reads;
modify the one or more on-target sequence reads to include the one or more intervals of sequence read data associated with the off-target sequence reads determined to be included in the on-target sequence reads; and
perform a genomic variant call utilizing the modified one or more on-target sequence reads.

22. (canceled)

23. A method, comprising:
receiving, at one or more processors, sequence read data for a sample derived from a subject;
determining, using the one or more processors, one or more on-target sequence reads and one or more off-target sequence reads based on the sequence read data;
identifying, using the one or more processors, and based on the one or more off-target sequence reads, one or more intervals of off-target sequence read data corresponding to an enhancer of zest homologs inhibitory protein (EZHIP);
modifying, using the one or more processors, the one or more on-target sequence reads to include the one or more intervals of off-target sequence read data corresponding to the EZHIP; and
performing, using the one or more processors, a genomic variant call utilizing the modified one or more on-target sequence reads.
